SEQUENCE LISTING (16 sequences)

SEQ ID NO: 1 - protein, 9 aa, Hepatitis C virus
LLPRRGPRL

SEQ ID NO: 2 - protein, 9 aa, Hepatitis C virus
WHYPCTVNF

SEQ ID NO: 3 - protein, 9 aa, Hepatitis C virus
LLSTTEWQI

SEQ ID NO: 4 - protein, 9 aa, Hepatitis C virus
ILPCSFTTL

SEQ ID NO: 5 - protein, 9 aa, Hepatitis C virus
ALYGVWPLL

SEQ ID NO: 6 - protein, 9 aa, Hepatitis C virus
YYKVFLARL

SEQ ID NO: 7 - protein, 9 aa, Hepatitis C virus
VFSDMETKL

SEQ ID NO: 8 - protein, 9 aa, Hepatitis C virus
AYSQQTRGL

SEQ ID NO: 9 - protein, 9 aa, Hepatitis C virus
ITYSTYCKF

SEQ ID NO: 10 - protein, 9 aa, Hepatitis C virus
RYAPACKPL

SEQ ID NO: 11 - protein, 9 aa, Hepatitis C virus
SMLTDPSHI

SEQ ID NO: 12 - protein, 9 aa, Hepatitis C virus
SYTWTGALI

SEQ ID NO: 13 - protein, 9 aa, Hepatitis C virus
ILMTHFFSI

SEQ ID NO: 14 - protein, 9 aa, Hepatitis C virus
FWAKHMWNF

SEQ ID NO: 15 - protein, 3010 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADMIMHTPGCVPCVRESNFSRCWVALTPTLAARNSSIPTTTIRRHVDLLVGAAALCSAMYVGDLCGSVFLVSQLFTFSPRRYETVQDCNCSIYPGHVSGHRMAWDMMMNWSPTTALVVSQLLRIPQAVVDMVAGAHWGVLAGLAYYSMVGNWAKVLIVMLLFAGVDGHTHVTGGRVASSTQSLVSWLSQGPSQKIQLVNTNGSWHINRTALNCNDSLQTGFIAALFYAHRFNASGCPERMASCRPIDEFAQGWGPITHDMPESSDQRPYCWHYAPRPCGIVPASQVCGPVYCFTPSPVVVGTTDRFGAPTYSWGENETDVLLLSNTRPPQGNWFGCTWMNSTGFTKTCGGPPCNIGGVGNNTLVCPTDCFRKHPEATYTKCGSGPWLTPRCMVDYPYRLWHYPCTVNFTVFKVRMYVGGVEHRLNAACNWTRGERCDLEDRDRSELSPLLLSTTEWQILPCSFTTLPALSTGLIHLHRNIVDVQYLYGIGSAVVSFAIKWEYILLLFLLLADARVCACLWMMLLIAQAEATLENLVVLNAASVAGAHGLLSFLVFFCAAWYIKGRLVPGAAYALYGVWPLLLLLLALPPRAYAMDREMAASCGGAVFVGLVLLTLSPYYKVFLARLIWWLQYFITRAEAHLQVWVPPLNVRGGRDAIILLTCAVHPELIFDITKLLLAILGPLMVLQAGITRVPYFVRAQGLIRACMLVRKVAGGHYVQMAFMKLAALTGTYVYDHLTPLRDWAHAGLRDLAVAVEPVVFSDMETKLITWGADTAACGDIISGLPVSARRGKEILLGPADSFGEQGWRLLAPITAYSQQTRGLLGCIITSLTGRDKNQVDGEVQVLSTATQSFLATCVNGVCWTVYHGAGSKTLAGPKGPITQMYTNVDQDLVGWPAPPGARSMTPCTCGSSDLYLVTRHADVVPVRRRGDSRGSLLSPRPISYLKGSSGGPLLCPSGHVVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGIEPNIRTGVRTITTGGPITYSTYCKFLADGGCSGGAYDIIICDECHSTDSTTILGIGTVLDQAETAGARLVVLATATPPGSITVPHPNIEEVALSNTGEIPFYGKAIPIEAIKGGRHLIFCHSKKKCDELAAKLTGLGLNAVAYYRGLDVSVIPTSGDVVVVATDALMTGFTGDFDSVIDCNTCVTQTVDFSLDPTFTIETTTLPQDAVSRAQRRGRTGRGRSGIYRFVTPGERPSGMFDSSVLCECYDAGCAWYELTPAETSVRLRAYLNTPGLPVCQDHLEFWESVFTGLTHIDAHFLSQTKQAGDNLPYLVAYQATVCARAQAPPPSWDQMWKCLIRLKPTLHGPTPLLYRLGAVQNEVTLTHPITKYIMACMSADLEVVTSTWVLVGGVLAALAAYCLTTGSVVIVGRIILSGRPAVIPDREVLYQEFDEMEECASHLPYIEQGMQLAEQFKQKALGLLQTATKQAEAAAPVVESKWRALEVFWAKHMWNFISGIQYLAGLSTLPGNPAIASLMAFTASITSPLTTQNTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLKDVWDWICTVLSDFKTWLQSKLLPRLPGLPFLSCQRGYKGVWRGDGIMQTTCPCGAQITGHVKNGSMRIVGPKTCSNTWHGTFPINAYTTGPCTPSPAPNYSRALWRVAAEEYVEVTRVGDFHYVTGMTTDNVKCPCQVPAPEFFTEVDGVRLHRYAPVCKPLLREEVVFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPIRAVEDEREISVPAEILRKPRKFPPALPIWARPDYNPPLLESWKDPDYVPPVVHGCPLPSTKAPPIPPPRRKRTVVLTESTVSSALAELATKTFGSSGSSAVDSGTATGPPDQASDDGDKGSDVESYSSMPPLEGEPGDPDLSDGSWSTVSGEAGEDVVCCSMSYTWTGALITPCAAEESKLPINPLSNSLLRHHSMVYSTTSRSASLRQKKVTFDRLQVLDDHYRDVLKEMKAKASTVKARLLSIEEACKLTPPHSAKSKFGYGAKDVRSLSSRAVNHIRSVWEDLLEDTETPIDTTIMAKNEVFCVQPEKGGRKPARLIVFPDLGVRVCEKMALYDVVSTLPQAVMGPSYGFQYSPGQRVEFLVNTWKSKKCPMGFSYDTRCFDSTVTENDIRTEESIYQCCDLAPEARQAIRSLTERLYVGGPLTNSKGQNCGYRRCRASGVLTTSCGNTLTCYLKATAACRAAKLQDCTMLVNGDDLVVICESAGTQEDAAALRAFTEAMTRYSAPPGDPPQPEYDLELITSCSSNVSVAHDASGKRVYYLTRDPTTPLARAAWETVRHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVKTKLKLTPIPAASQLDLSGWFVAGYNGGDIYHSLSRARPRWFMLCLLLLSVGVGIYLLPNR

SEQ ID NO: 16 - protein, 3010 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHAPGCVPCVRENNSSRCWVALTPTLAARNASVPTTTLRRHVDLLVGTAAFCSAMYVGDLCGSVFLISQLFTFSPRRHETVQDCNCSIYPGHVSGHRMAWDMMMNWSPTAALVVSQLLRIPQAVMDMVAGAHWGVLAGLAYYSMVGNWAKVLIVMLLFAGVDGHTRVTGGVQGHVTSTLTSLFRPGASQKIQLVNTNGSWHINRTALNCNDSLKTGFLAALFYTHKFNASGCPERMASCRSIDKFDQGWGPITYAQPDNSDQRPYCWHYAPRQCGIVPASQVCGPVYCFTPSPVVVGTTDRFGAPTYNWGDNETDVLLLNNTRPPHGNWFGCTWMNSTGFTKTCGGPPCNIRGVGNNTLTCPTDCFRKHPDATYTKCGSGPWLTPRCLVDYPYRLWHYPCTVNFTIFKVRMYVGGVEHRLDAACNWTRGERCDLEDRDRAELSPLLLSTTEWQILPCSYTTLPALSTGLIHLHQNIVDIQYLYGIGSAVVSIAIKWEYVVLLFLLLADARVCACLWMMLLIAQAEAALENLVVLNAASVVGAHGMLPFFMFFCAAWYMKGRLVPGAAYAFYGVWPLLLLLLALPPRAYAMDREMVASCGGGVFVGLALLTLSPYCKVFLARLIWWLQYFITKAEAHLQVSLPPLNVRGGRDAIILLMCAVHPELIFDITKLLLSILGPLMVLQASLIRVPYFVRAQGLIRACMLVRKAAGGHYVQMAFVKLAALTGTYVYDHLTPLQDWAHVGLRDLAVAVEPVVFSAMETKVITWGADTAACGDIISGLPVSARRGKEILLGPADSFEGQGWRLLAPITAYSQQTRGLLGCIITSLTGRDKNQVEGEVQVVSTAKQSFLATCVNGACWTVFHGAGSKTLAAAKGPITQMYTNVDQDLVGWPAPPGARSLTPCTCGSSDLYLVTRHADVIPVRRRGDSRGSLLSPRPISYLKGSSGGPLLCPSGHVVGIFRAAVCTRGVAKAVDFIPVESMETTMRSPVFTDNSTPPAVPQTFQVAHLHAPTGSGKSTKVPAAYAAQGYMVLVLNPSVAATLGFGAYMSKAHGIDPNIRTGVRTITTGAPITYSTYGKFLADGGCSGGAYDIIICDECHSTDSTSILGIGTVLDQAETVGARFVVLATATPPGSITFPHPNIEEVPLANTGEIPFYAKTIPIEVIRGGRHLIFCHSKKKCDELPAKLSALGLNAVAYYRGLDVSVIPASGDVVVVATDALMTGFTGDFDSVIDCNTCVTQTVDFSLDPTFTIETTTVPQDAVSRTQRRGRTGRGRRGIYRFVTPGERPSAMFDSSVLCECYDAGCAWYELTPAETSVRLRAYLNTPGLPVCQDHLEFWESVFTGLTHIDAHFLSQTKQAGDNFPYLVAYQATVCARAKAPPPSWDQMWKCLIRLKPTLHGPTPLLYRLGAVQNEVTLTHPITKYIMACMSADLEVVTSTWVLVGGVLAALAAYCLTTGSVVIVGRIILSGRPAVIPDREVLYQEFDEMEECASHLPYIEQGMQLAEQFKQKALGLLQTATKQAEAAAPVVESKWRALETFWAKHMWNFISGIQYLAGLSTLPGNPAIASLMAFTASITSPLATQYTLLFNILGGWVAAQLAPPSAASAFVGAGIAGAAVGSIGLGKVLVDILAGYGAGVAGALVAFKVMSGDMPSTEDLVNLLPAILSPGALVVGVVCAAILRRHVGPGEGAVQWMNRLIAFASRGNHVSPTHYVPESDAAARVTQILSNLTITQLLKRLHQWINEDCSTPCSGSWLRDVWDWICTVLADFKTWLQSKLLPRLPGVPFFSCQRGYKGVWRGDGIMYTTCPCGAQITGHVKNGSMRIVGPRTCSNTWHGTFPINAYTTGPCTPSPAPNYSRALWRVAAEEYVEVTRVGDFHYVTGMTTDNVKCPCQVPAPEFFTELDGVRLHRYAPACKPLLRDEVTFQVGLNQYTVGSQLPCEPEPDVTVVTSMLTDPSHITAEAARRRLARGSPPSLAGSSASQLSALSLKATCTTHHGAPDTDLIEANLLWRQEMGGNITRVESENKIVILDSFEPLRAEEDEREVSAAAEILRKTRKFPAAMPVWARPDYNPPLLESWKNPDYVPPVVHGCPLPPTKAPPIPPPRRKRTVVLTESTVSSALAELATKTFGGSGSSAVDSGTATGPPDQASAEGDAGSDAESYSSMPPLEGEPGDPDLSDGSWSTVSEEASEDVVCCSMSYTWTGALITPCAAEESKLPINALSNPLLRHHNMVYSTTSRSASLRQKKVTFDRMQVLDDHYRDVLKEMKAKASTVKAKLLSVEEACKLTPPHSAKSKFGYGAKDVRSLSSRAVNHIRSVWKDLLEDTDTPIQTTIMAKNEVFCVQPEKGGRKPARLIVFPDLGVRVCEKMALYDVVSTLPQAVMGSSYGFQYSPKQRVEFLVNTWKAKKCPMGFSYDTRCFDSTVTENDIRVEESIYQCCDLAPEARQAIRSLTERLYIGGPMTNSKGQNCGYRRCRASGVLTTSCGNTLTCYLKAAAACRAAKLQDCTMLVCGDDLVVICDSAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGATYSIEPLDLPQIIQRLHGLSAFSLHSYSPGEINRVASCLRKLGVPPLRVWRHRARSVRAKLLSQGGRAATCGKYLFNWAVKTKLKLTPIPEASQLDLSGWFVAGYSGGDIYHSLSRARPRWFMWCLLLLSVGVGIYLLPNR

(19) United States
(12) Patent Application Publication
(10) Pub. No.: US 2011/0206711 A1
(43) Pub. Date: Aug. 25, 2011

(54) METHOD FOR INDUCING CYTOTOXIC T-CELLS, CYTOTOXIC T-CELL INDUCER, AND PHARMACEUTICAL COMPOSITION AND VACCINE EMPLOYING SAME

(75) Inventors: Tomoya Miyakawa, Tokyo (JP); Keiko Udaka, Kochi (JP); Morikazu Onji, Ehime (JP)

(73) Assignees: NEC CORPORATION, Tokyo (JP); KOCHI UNIVERSITY, Kochi (JP); EHIME UNIVERSITY, Ehime (JP)

(21) Appl. No.: 12/279,556

(22) PCT Filed: Feb. 16, 2007
(86) PCT No.: PCT/JP2007/000095

§ 371 Date: Aug. 15, 2008

(30) Foreign Application Priority Data
Feb. 17, 2006 (JP) 2006-041508

(51) Int. Cl. A61K 39/29; C07K 7/06; C12N 5/00; A61K 38/08; A61P 31/14

(52) U.S. Cl. 424/189.1; 530/327; 530/328; 435/375; 514/4.3

(57) ABSTRACT

A method for inducing cytotoxic T-cells that includes binding to an HLA molecule on the surface of a cell that is a target of a cytotoxic T-cell a peptide containing one or more types of amino acid sequence selected from the group consisting of SEQ ID NOS: 1 to 13 and consisting of not less than 8 and not more than 11 amino acid residues, or a peptide derived from a precursor thereof.

### TECHNICAL FIELD

The present invention relates to a method for inducing cytotoxic T-cells, a cytotoxic T-cell inducer, and a pharmaceutical composition and a vaccine employing same.

### BACKGROUND ART

When infection with a virus such as a hepatitis C virus (HCV) occurs, a virus elimination reaction due to natural immunity proceeds, a specific immune response is subsequently induced, and a virus elimination reaction proceeds.

In the specific immune response, virus in a body fluid is eliminated by a neutralizing antibody, and intracellular virus is eliminated by cytotoxic T-cells (CTL). That is, the CTL specifically recognizes a virus antigen peptide (CTL epitope) consisting of 8 to 11 amino acids presented in an HLA class I molecule on the surface of an infected cell, and eliminates the virus by damaging the infected cell. Identifying such a virus-specific CTL epitope is therefore important when investigating the specific immune response.

Identification of this CTL epitope, and methods for ascertaining immunity-inducing capability, are described in Non-patent Publications 1 to 4.
[Non-patent Publication 1] Battergay, M., J. Fikes, et al. (1995). ‘Patients with Chronic Hepatitis C Have Circulating Cytotoxic T Cells Which Recognize Hepatitis C Virus-Encoded Peptides Binding to HLA-A2.1 Molecules.’ J Virol 69: pp. 2462-2470
[Non-patent Publication 2] Cerny, A., J. McHutchinson, et al. (1995). ‘Cytotoxic T Lymphocyte Response to Hepatitis C Virus-derived Peptides Containing the HLA A2.1 Binding Motif.’ J Clin Invest 95: pp. 521-530
[Non-patent Publication 3] Kurokohchi, K., K. Arima, et al. (2001). ‘A novel cytotoxic T-cell epitope presented by HLA-A24 molecule in hepatitis C virus infection.’ J Hepatology 34: pp. 930-935
[Non-patent Publication 4] Nakamoto, Y., S. Kaneko, et al. (2003). ‘Analysis of the CD8-Positive T Cell Response in Japanese Patients With Chronic Hepatitis C Using HLA-A*2402 Peptide Tetramers.’ J Med Virol 70: pp. 51-61

### DISCLOSURE OF THE INVENTION

### Problems to be Solved by the Invention

The above publications cite HLA-binding peptides with a 2 to 5 sequence peptides that induce cytotoxic T-cells. On the other hand, it is known that hepatitis C virus mutates frequently once it has entered the human body. Therefore, it could well happen that immunotherapy using peptides described in the above publications may not be effective to such mutated viruses. In order to treat and prevent a disease caused by hepatitis C virus, it is desired that more peptides are identified in order to increase the option of choosing alternative peptides which have the capability of inducing cytotoxic T-cells.

The present invention has been accomplished in the light of the above-mentioned circumstances, and it is an exemplary object thereof to provide a method for inducing cytotoxic T-cells, a cytotoxic T-cell inducer, and a pharmaceutical composition and a vaccine employing same, that enable a disease that is caused by hepatitis C virus to be treated and prevented effectively.

### Means for Solving the Problems

In one exemplary aspect of the invention, there is provided a method for inducing cytotoxic T-cells, the method including binding, to an HLA molecule on the surface of a cell that is a target of a cytotoxic T-cell, a peptide containing one or more types of amino acid sequence selected from the group consisting of SEQ ID NOS: 1 to 13 and consisting of not less than 8 and not more than 11 amino acid residues, or a peptide derived from a precursor thereof.

Furthermore, in another exemplary aspect of the invention, there is provided a cytotoxic T-cell inducer containing at least one of a peptide and a precursor thereof, the peptide containing one or more types of amino acid sequence selected from the group consisting of SEQ ID NOS: 1 to 13, consisting of not less than 8 and not more than 11 amino acid residues, and binding to an HLA molecule on the surface of a cell that is a target of a cytotoxic T-cell.

Moreover, in another exemplary aspect of the invention, there is provided a pharmaceutical composition for the treatment of a disease that is caused by hepatitis C virus, the composition containing at least one of a peptide and a precursor thereof, the peptide containing one or more types of amino acid sequence selected from the group consisting of SEQ ID NOS: 1 to 13, consisting of not less than 8 and not more than 11 amino acid residues, and binding to an HLA molecule on the surface of a cell infected with hepatitis C virus.

Furthermore, in another exemplary aspect of the invention, there is provided a vaccine used for the prevention or treatment of a disease that is caused by hepatitis C virus, the vaccine containing at least one of a peptide and a precursor thereof, the peptide containing one or more types of amino acid sequence selected from the group consisting of SEQ ID NOS: 1 to 13, consisting of not less than 8 and not more than 11 amino acid residues, and binding to an HLA molecule on the surface of a cell infected with hepatitis C virus.

### Effects of the Invention

In an exemplary advantage according to the invention, since cytotoxic T-cells can be induced effectively, a pharmaceutical composition and a vaccine that are useful in particular for the treatment or prevention of a disease that is caused by hepatitis C virus can be obtained.

### BRIEF DESCRIPTION OF THE DRAWINGS

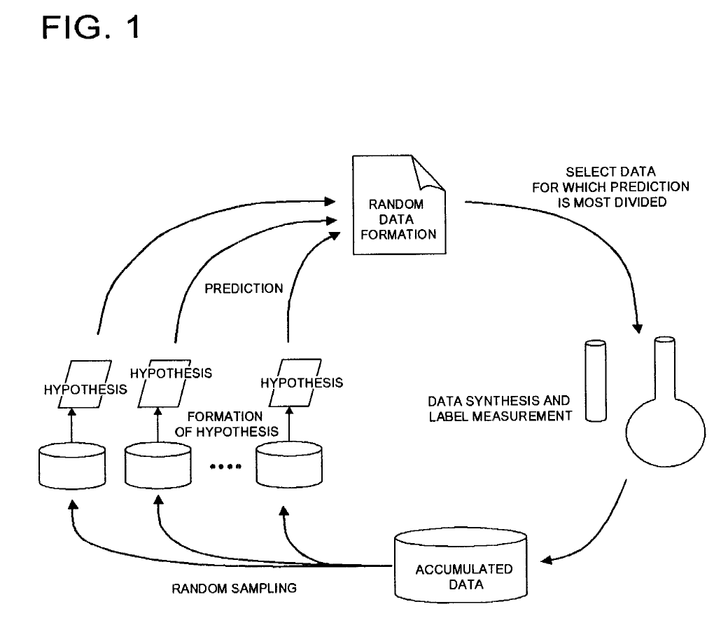

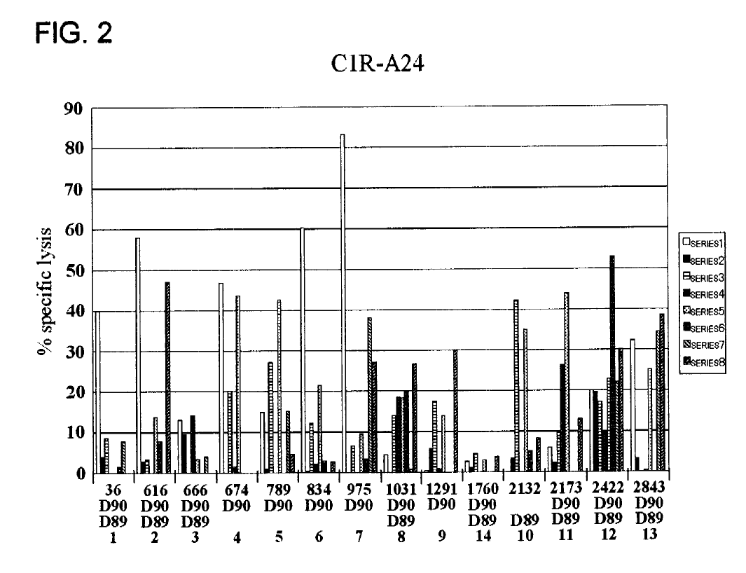

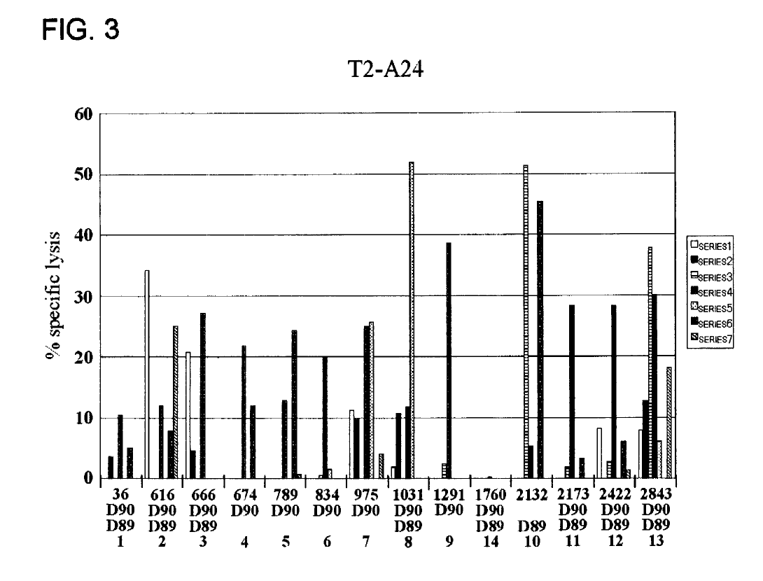

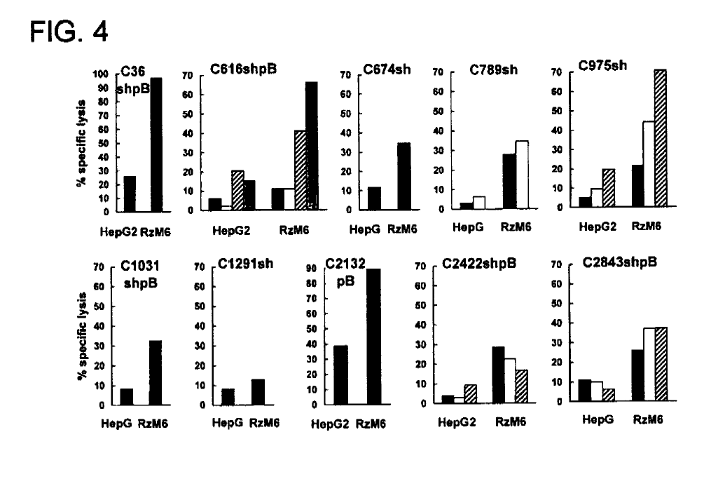

The above-mentioned object, other objects, features, and advantages will become more apparent from exemplary embodiments explained below by reference to the attached drawings.

[FIG. 1] A schematic drawing for explaining an active learning experiment design used in an example.

[FIG. 2] A graph showing the results of an investigation of cytotoxic T-cell-inducing capability in an example.

[FIG. 3] A graph showing the results of an investigation of cytotoxic T-cell-inducing capability in an example.

[FIG. 4] A graph showing the results of an investigation of damage to hepatitis C virus-expressing cells in an example.

### EXEMPLARY EMBODIMENT

Exemplary embodiments for carrying out the present invention are explained below using the drawings.

### A First Exemplary Embodiment

The method for inducing cytotoxic T-cells related to the present exemplary embodiment includes binding, to an HLA molecule on the surface of a cell that is a target of a cytotoxic T-cell, a peptide containing one or more types of amino acid sequence selected from the group consisting of SEQ ID NOS: 1 to 13 and consisting of not less than 8 and not more than 11 amino acid residues, or a peptide derived from a precursor thereof (hereinafter, called an ‘HLA-binding peptide’).

All of the amino acid sequences shown in SEQ ID NOS: 1 to 13 are sequences consisting of 9 amino acid residues contained in a certain genome protein of a hepatitis C virus (HCV) shown in SEQ ID NOS: 15 and 16.

Furthermore, among amino acid sequences for which the binding to an HLA molecule, predicted by a hypothesis obtained using an active learning experiment method (Japanese Patent Application Laid-open No. H11-316754 (1999)), is 3 or greater in terms of a −log Kd value, these amino acid sequences have actually been confirmed by an HLA molecule binding experiment to exhibit HLA binding and have been shown to be capable of inducing cytotoxic T-cells.

When selecting a candidate, an amino acid sequence for which the binding to an HLA molecule in terms of a −log Kd value is 3 or greater is selected from the viewpoint that in the field of biochemistry it is known that a binding ability, in terms of a −log Kd value, of about 3 can be treated as the threshold level for whether or not a peptide actually binds to an MHC, which includes an HLA.

That is, peptides having amino acid sequences shown in SEQ ID NOS: 1 to 13 correspond to an epitope of an antigen peptide presented in an HLA molecule on the surface of a cell infected with hepatitis C virus.

It has been confirmed that peptides having the amino acid sequences shown in SEQ ID NOS: 1 to 13 bind to a product (HLA-A24 molecule) of the HLA-A*2402 gene, which is one of the alleles of gene A, and about 50% of Japanese people have an HLA-A24 molecule of a subclass of the gene A.

The sequences of SEQ ID NOS: 1 to 13 and the sequence of SEQ ID NO: 14 for comparison therewith are shown in Table 1 below.

TABLE 1

| | | | | Binding |
|---|---|---|---|---|
| SEQ ID | | Predicted | | Experiment |
| No | SEQ | Score | SEQ Name | Data |
| 1 | LLPRRGPRL | 5.30 | 36D90D89 | 7.71 |
| 2 | WHYPCTVNF | 5.66 | 616D90D89 | 6.39 |
| 3 | LLSTTEYQI | 5.35 | 666D90D89 | 8.34 |
| 4 | ILPCSFTTL | 6.75 | 674D90 | 7.66 |
| 5 | ALYGVYPLL | 5.80 | 789D90 | 6.98 |
| 6 | YYKVFLARL | 5.49 | 834D90 | 7.12 |
| 7 | VFSDMETKL | 5.54 | 975D90 | 7.31 |
| 8 | AYSQQTRGL | 5.76 | 1031D90D89 | 6.54 |
| 9 | ITYSTYCKF | 5.27 | 1291D90 | 6.98 |
| 10 | RYAPACKPL | 5.47 | 2132D89 | 6.76 |
| 11 | SMLTDPSHI | 5.55 | 2173D90D89 | 6.94 |
| 12 | SYTWTGALI | 5.51 | 2422D90D89 | 7.13 |
| 13 | ILMTHFFSI | 5.57 | 2843D90D89 | 7.90 |
| 14 | FWAKMMYNF | 5.93 | 1760D90D89 | 8.10 |

Among the sequences of SEQ ID NOS: 1 to 14, those having a sequence name with ‘D90’ denote sequences consisting of 9 amino acid residues contained in a certain genome protein (SEQ ID NO: 15) of HCV D90208 strain, which will be described later. Furthermore, those having a sequence name with ‘D89’ denote sequences consisting of 9 amino acid residues contained in a certain genome protein (SEQ ID NO: 16) of HCV D89815 strain, which will be described later. Moreover, those with ‘D90D89’ denote sequences contained in common in both of the above. Furthermore, in the table, predicted scores and binding experiment data with respect to binding to the HLA-A24 molecule of each sequence are shown in terms of −log Kd values. As can be seen, a correlation exists between the predicted score and the binding experiment data.

Since there is no conventional technique for discovering an HLA-binding peptide by utilizing such an experimental design method, there are only a very small number of HLA-binding peptides that have been experimentally confirmed to have HLA-binding properties. Because of this, even when a peptide consisting of 9 amino acid residues is randomly synthesized by a conventional method and subjected to an experiment to find out if it binds to an HLA molecule, there is a probability of only about 1 in 100 of finding one that has a binding, in terms of a −log Kd value, exceeding 6.

Furthermore, among candidates predicted by the active learning experiment method as described later, a peptide having the amino acid sequence of SEQ ID NO: 14 showed high binding in an HLA molecule binding experiment but did not show a capability of inducing cytotoxic T-cells. On the other hand, the peptides having the amino acid sequences of SEQ ID NOS: 1 to 13 showed a capability of inducing cytotoxic T-cells.

As described above, in order to induce cytotoxic T-cells, not only predicting the sequence of an HLA-binding peptide by the active learning experiment method but also selecting a specific amino acid sequence among those predicted candidates are important factors.

In the present exemplary embodiment, a peptide having an amino acid sequence of SEQ ID NOS: 1 to 13 is introduced into a system containing cells that are the target of cytotoxic T-cells (CTL), binds to an HLA class I molecule on the surface of the cell, and is presented to the cytotoxic T-cell as an antigen peptide. The cytotoxic T-cell specifically recognizes this and is induced. Furthermore, the induced cytotoxic T-cell damages the cell that presents the antigen peptide. In this way, the peptide having an amino acid sequence of SEQ ID NOS: 1 to 13 functions as a virus antigen (CTL epitope) for inducing the cytotoxic T-cell.

The ‘inducing’ referred to here means generating an activity or an action from a material or a state in which there is almost no activity or action. In particular, ‘inducing a cytotoxic T-cell’ means differentiating the cytotoxic T-cells that specifically recognize a certain antigen into effector cells having the capability of killing target cells, and/or proliferating the cytotoxic T-cells, in vitro or in vivo.

From another viewpoint, by containing at least one of a peptide and a precursor thereof, the peptide containing one or more types of amino acid sequence selected from the group consisting of SEQ ID NOS: 1 to 13, consisting of not less than 8 and not more than 11 amino acid residues, and binding to an HLA molecule on the surface of a cell that is a target of a cytotoxic T-cell, a cytotoxic T-cell inducer can be obtained.

The ‘cytotoxic T-cell inducer’ means a drug that exhibits an action of changing a state in which CD8 positive T-cells specifically recognizing a certain antigen are not present, or are present only at a very low proportion, into a state in which cytotoxic T-cells recognizing this antigen are present at a very high proportion, and an action of enhancing the capability of individual cytotoxic T-cells of killing a target.

Furthermore, among HLA-binding peptides containing an amino acid sequence that is predicted by the active learning experiment method and confirmed by the binding experiment to actually bind, since only some thereof induced cytotoxic T-cells, it is surmised that, although it remains important for a peptide to bind to an HLA molecule on the surface of the cell in order to induce cytotoxic T-cells, a difference in T-cell repertoire between individuals and the like or another factor is acting.

HLA-binding peptides used in the present exemplary embodiment include those formed by modifying a sequence or a site that does not affect the binding to an HLA molecule in a peptide having a sequence of SEQ ID NOS: 1 to 13 by chemical modification or substitution with a different stereoisomer of an amino acid used in the HCV genome protein. Furthermore, such an HLA peptide may be a peptide consisting of amino acid residues alone as described above, but it is not particularly limited thereto. For example, it may be an HLA-binding peptide precursor that is optionally modified with a sugar chain or a fatty acid group and the like as long as the effects of the present invention are not impaired. Such a precursor is subjected to a change involving digestion by a proteolytic enzyme and the like in a living mammalian body such as in a human digestive organ to become an HLA-binding peptide, thus exhibiting similar effects to those shown by the above-mentioned HLA-binding peptide.

In the present exemplary embodiment, since cytotoxic T-cells can be induced by a peptide that binds to the HLA-A24 molecule, which is often seen in Asian people, such as Japanese people, this can be utilized in the development of a therapeutic drug, a prophylactic drug, and the like that is particularly effective for Asian people, such as Japanese people. The proportion of people having the HLA-A24 molecule is as low as 10 or so percent in Europe and America, but it is still one of the main HLA genotypes, and it is important as a target molecule for an immunotherapeutic drug.

Moreover, the HLA-binding peptides used in the present exemplary embodiment may be produced using a method known to a person skilled in the art. For example, they may be artificially synthesized by a solid-phase method or a liquid-phase method. Alternatively, these HLA-binding peptides may be produced by expressing them from a DNA fragment or a recombinant vector coding for these HLA-binding peptides. These HLA-binding peptides thus obtained can be identified by a method known to a person skilled in the art. For example, identification is possible by use of Edman degradation, mass spectrometry, and the like.

### A Second Exemplary Embodiment

The pharmaceutical composition related to the present exemplary embodiment contains the cytotoxic T-cell inducer explained in Embodiment 1. That is, this pharmaceutical composition is a pharmaceutical composition for the treatment of a disease that is caused by hepatitis C virus, the composition containing at least one of a peptide and a precursor thereof, the peptide containing one or more types of amino acid sequence selected from the group consisting of SEQ ID NOS: 1 to 13, consisting of not less than 8 and not more than 11 amino acid residues, and binding to an HLA molecule on the surface of a cell expressing hepatitis C virus (hereinafter, called an ‘expressing cell’).

The peptide having an amino acid sequence of SEQ ID NOS: 1 to 13, as explained in Embodiment 1, has HLA-binding properties and can induce cytotoxic T-cells.

In the explanation of Embodiment 1, cytotoxic T-cells can be induced by using the expressing cells as the target cells for the cytotoxic T-cells, and the expressing cells are damaged by these induced cytotoxic T-cells.

That is, by administering the pharmaceutical composition of the present exemplary embodiment to a patient with a disease that is caused by hepatitis C virus, the peptide contained in the composition binds to an HLA class I molecule on the surface of cells within the patient's body, and is presented as an antigen peptide to cytotoxic T-cells. The cytotoxic T-cells specifically recognize this and are activated. In this way, the cytotoxic T-cells are induced. Furthermore, the induced cytotoxic T-cells damage the infected cells presenting the antigen peptide, and this function enables a contribution to be made to the treatment of hepatitis C.

The HLA-binding peptide contained in the present exemplary embodiment may be a peptide consisting of amino acid residues alone as described above, but it is not particularly limited thereto. For example, it may be an HLA-binding peptide precursor that is optionally modified with a sugar chain or a fatty acid group and the like as long as the effects of the present invention are not impaired. Such a precursor is subjected to a change involving digestion by a proteolytic enzyme and the like in a living mammalian body such as in a human digestive organ to become an HLA-binding peptide, thus exhibiting similar effects to those shown by the above-mentioned HLA-binding peptide. Furthermore, such HLA-binding peptides may be produced by a method known to a person skilled in the art. For example, they may be artificially synthesized by a solid phase method or a liquid phase method.

The pharmaceutical composition of the present exemplary embodiment may be administered to a patient by dissolving it in a water-soluble solvent and made into a preparation in the form of a pharmaceutically acceptable salt.

Examples of the form of such a pharmaceutically acceptable salt include water-soluble salts that are physiologically acceptable, such as sodium, potassium, magnesium, and calcium salts that are buffered at a physiological pH. Other than the water-soluble solvent, a water-insoluble solvent may be used, and examples of such a water-insoluble solvent include alcohols such as ethanol and propylene glycol.

A preparation containing the pharmaceutical composition of the present exemplary embodiment may contain agents for various purposes, and examples of such agents include a preservative and a buffer agent.

Examples of the preservative include sodium bisulfite, sodium bisulfate, sodium thiosulfate benzalkonium chloride, chlorobutanol, thimerosal, phenylmercuric acetate, phenylmercuric nitrate, methylparaben, polyvinyl alcohol, phenylethyl alcohol, ammonia, dithiothreitol, and β-mercaptoethanol. Examples of the buffer agent include sodium carbonate, sodium borate, sodium phosphate, sodium acetate, and sodium bicarbonate. These agents can be present in an amount that enables the pH of the system to be maintained at between 2 and 9, and preferably at between 4 and 8.

### A Third Exemplary Embodiment

A vaccine related to the present exemplary embodiment contains the pharmaceutical composition explained in the second exemplary embodiment. That is, this vaccine is a vaccine used for the prevention or treatment of a disease that is caused by hepatitis C virus, the vaccine containing at least one of a peptide and a precursor thereof, the peptide containing one or more types of amino acid sequence selected from the group consisting of SEQ ID NOS: 1 to 13, consisting of not less than 8 and not more than 11 amino acid residues, and binding to an HLA molecule on the surface of a cell expressing hepatitis C virus (hereinafter, called an ‘expressing cell’).

The peptide having an amino acid sequence of SEQ ID NOS: 1 to 13 contained in the pharmaceutical composition, as explained in the first exemplary embodiment, has HLA-binding properties and can induce cytotoxic T-cells.

As in the second exemplary embodiment, in the explanation of the first exemplary embodiment, cytotoxic T-cells can be induced by using the HCV expressing cells as the target cells for the cytotoxic T-cells, and the HCV expressing cells are damaged by these induced cytotoxic T-cells.

That is, by administering the vaccine of the present exemplary embodiment to a patient with hepatitis C virus, the peptide contained in the composition binds to an HLA-A24 molecule on the surface of tissue cells of the patient, including dendritic cells always present at an injection site. When cytotoxic T-cells specific to the peptide recognize it, they are activated, proliferate, and circulate via general circulation. When cytotoxic T-cells specific to the peptide enter the expressing liver tissue, they recognize the same virus-derived peptide naturally binding to an HLA-A24 molecule present on the surface of hepatitis C virus-expressing liver cells, and kill the expressing cells, thus cutting off the virus source. This function enables a contribution to be made to the treatment of a disease that is caused by hepatitis C virus.

Alternatively, by administering the vaccine of the present exemplary embodiment to a healthy human body, cytotoxic T-cells are induced, the induced cytotoxic T-cells build up within the body, and when hepatitis C virus enters, cells expressing this hepatitis C virus can be killed. This function enables a contribution to be made to the prevention of a disease that is caused by hepatitis C virus.

The HLA-binding peptide contained in the present exemplary embodiment may be a peptide consisting of amino acid residues alone as described above, but it is not particularly limited thereto. For example, it may be an HLA-binding peptide precursor that is optionally modified with a sugar chain or a fatty acid group and the like as long as the effects of the present invention are not impaired. Such a precursor is subjected to a change involving digestion by a proteolytic enzyme and the like in a living mammalian body such as in a human digestive organ to become an HLA-binding peptide, thus exhibiting similar effects to those shown by the above-mentioned HLA-binding peptide. Furthermore, such HLA-binding peptides may be produced by a method known to a person skilled in the art. For example, they may be artificially synthesized by a solid phase method or a liquid phase method.

Furthermore, the vaccine of the present exemplary embodiment may be used in the form of an inactive component-containing vaccine that contains a component, other than components of the pharmaceutical composition, that has no activity itself but has an effect in yet further enhancing the effect of the pharmaceutical composition as a vaccine. Examples of the inactive component include an adjuvant and a toxoid.

The pharmaceutical composition of the second exemplary embodiment and the vaccine of the third exemplary embodiment are internally administered by injection or infusion via subcutaneous, intravenous, or intramuscular administration, and the like, by percutaneous administration, or by inhalation via the mucosa of the nose, throat, and the like.

The amount thereof per administration may be set between an amount that can significantly induce cytotoxic T-cells and an amount that does not damage a significant number of uninfected cells.

Exemplary embodiments of the present invention are described above, but are exemplifications of the present invention, and various constitutions other than those above may be employed.

### EXAMPLE 1

The present invention is further explained below by reference to Examples, but the present invention is not limited thereto.

Specifically, procedures of prediction, experiment, and evaluation in the present Examples were carried out based on an active learning experiment design described in WO2006/004182, and in general the following steps were repeated, thus creating rules. A schematic drawing for the active learning experiment design employed here is shown in FIG. 1.
(1) A trial of a lower-order learning algorithm, which will be described later, was carried out once. That is, a plurality of hypotheses were generated by random sampling from accumulated data and, with regard to randomly expressed candidate query points (peptides), a point that showed the largest distribution of predicted values was selected as a query point to be subjected to an experiment.
(2) The peptide at the selected query point was prepared by a synthesis and purification method, which will be described later, and the actual binding ability was measured by an experiment, which will be described later, and added to accumulated data.

According to such an active learning method, the number of repetitions of the binding experiment for peptides consisting of 9 amino acid residues, which would otherwise have to be carried out for the 500,000,000,000 (=20⁹) or more combinations of all the candidates for HLA-binding peptides, could be reduced.

Amino acid sequences shown in SEQ ID NOS: 1 to 14 were extracted by the rules explained above.

### <Synthesis and Purification of Peptide>

A peptide having an amino acid sequence of SEQ ID NOS: 1 to 14 was manually synthesized by the Merrifield solid-phase method using Fmoc amino acids. After deprotection, reverse phase HPLC purification was carried out using a C18 column to give a purity of 95% or higher. Identification of the peptide and confirmation of its purity were carried out using a MALDI-TOF mass spectrometer (Voyager DE RP, PerSeptive). Quantitative analysis of the peptide was carried out by a Micro BCA assay (Pierce Corp.) using BSA as a standard protein.

### <Experiment of Binding Peptide to HLA-A24 Molecule>

The ability of a peptide having an amino acid sequence of SEQ ID NOS: 1 to 14 to bind to an HLA-A24 molecule, which is a product of the HLA-A*2402 gene, was measured using C1R-A24 cells expressing the HLA-A24 molecule (cells produced by associate professor Masafumi Takiguchi, Kumamoto University being supplied with permission by Assistant Professor Masaki Yasukawa, Ehime University).

C1R-A24 cells were first exposed to acidic conditions at a pH of 3.3 for 30 seconds, thus dissociating and removing a light chain β2m, which is associated with HLA class I molecules in common, and an endogenous peptide originally bound to the HLA-A*2402 molecule. After neutralization, purified β2m was added to C1R-A24 cells, the obtained product was added to serial dilutions of a peptide, and incubated on ice for 4 hours. Staining was carried out using fluorescently labeled monoclonal antibody 17A12, which recognizes association (MHC-pep) of the three members, that is, the HLA-A*2402 molecule, the peptide, and β2m, which had reassociated during the incubation.

Subsequently, the MHC-pep count per C1R-A24 cell (proportional to the strength of fluorescence of the above-mentioned fluorescent antibody) was quantitatively measured using a FACScan fluorescence-activated flow cytometer (Becton Dickinson Biosciences). A binding dissociation constant Kd value between the HLA-A24 molecule and the peptide was calculated from the average strength of fluorescence per cell by a published method (Udaka et al., Immunogenetics, 51, 816-828, 2000).

<Evaluation Results from Binding Experiment>

The prediction results and the experimental results shown in Table 1 above were obtained.

The sequences of SEQ ID NOS: 1 to 3, the sequence of SEQ ID NO: 8, and the sequences of SEQ ID NOS: 11 to 14 in Table 1 are sequences consisting of 9 amino acid residues contained in common in both the full-length sequence (SEQ ID NO: 15) of a certain genome protein of HCV D90208 strain registered in GENBANK and the full-length sequence (SEQ ID NO: 16) of a certain genome protein of HCV D89815 strain similarly registered in GENBANK.

Furthermore, the sequences of SEQ ID NOS: 4 to 7 and the sequence of SEQ ID NO: 9 are sequences consisting of 9 amino acid residues contained only in the full-length sequence (SEQ ID NO: 15) of the certain genome protein of the HCV D90208 strain. Moreover, SEQ ID NO: 10 is a sequence consisting of 9 amino acid residues contained only in the full-length sequence (SEQ ID NO: 16) of the certain genome protein of the HCV D89815 strain.

The full-length amino acid sequence of the certain genome protein of the HCV D90208 strain is shown in SEQ ID NO: 15

(MSTNPKPQRKTKRNTNRRPQDVKFPGGGQIVGGVYLLPRRGPRLGVR
ATRKTSERSQPRGRRQPIPKARRPEGRTWAQPGYPWPLYGNEGMGWAG
WLLSPRGSRPSWGPTDPRRRSRNLGKVIDTLTCGFADLMGYIPLVGAP
LGGAARALAHGVRVLEDGVNYATGNLPGCSFSIFLLALLSCLTIPASA
YEVRNVSGIYHVTNDCSNSSIVYEAADMIMHTPGCVPCVRESNFSRCW
VALTPTLAARNSSIPTTTIRRHVDLLVGAAALCSAMYVGDLCGSVFLV
SQLFTFSPRRYETVQDCNCSIYPGHVSGHRMAWDMMMNWSPTTALVVS
QLLRIPQAVVDMVAGAHWGVLAGLAYYSMVGNWAKVLIVMLLFAGVDG
HTHVTGGRVASSTQSLVSWLSQGPSQKIQLVNTNGSWHINRTALNCND
SLQTGFIAALFYAHRFNASGCPERMASCRPIDEFAQGWGPITHDMPES
SDQRPYCWHYAPRPCGIVPASQVCGPVYCFTPSPVVVGTTDRFGAPTY
SWGENETDVLLLSNTRPPQGNWFGCTWMNSTGETKTCGGPPCNIGGVG
NNTLVCPTDCFRKHPEATYTKCGSGPWLTPRCMVDYPYRLWHYPCTVN
FTVFKVRMYVGGVEHRLNAACNWTRGERCDLEDRDRSELSPLLLSTTE
WQILPCSFTTLPALSTGLIHLHRNIVDVQYLYGIGSAVVSFAIKWEYI
LLLFLLLADARVCACLWMMLLIAQAEATLENLVVLNAASVAGAHGLLS
FLVFFCAAWYIKGRLVPGAAYALYGVWPLLLLLLALPPRAYANDREMA
ASCGGAVFVGLVLLTLSPYYKVFLARLIWWLQYFITRAEAHLQVWVPP
LNVRGGRDAIILLTCAVHPELIFDITKLLLAILGPLMVLQAGITRVPY
FVRAQGLIRACMLVRKVAGGHYVQMAFMKLAALTGTYVYDHLTPLRDW
AHAGLRDLAVAVEPVVFSDMETKLITWGADTAACGDIISGLPVSARRG
KEILLGPADSFGEQGWRLLAPITAYSQQTRGLLGCIITSLTGRDKNQV
DGEVQVLSTATQSFLATCVNGVCWTVYHGAGSKTLAGPKGPITQMYTN
VDQDLVGWPAPPGARSMTPCTCGSSDLYLVTRHADVVPVRRRGDSRGS
LLSPRPISYLKGSSGGPLLCPSGHVVGIFRAAVCTRGVAKAVDFIPVE
SMETTMRSPVFTDNSSPPAVPQTFQVAHLHAPTGSGKSTKVPAAYAAQ
GYKVLVLNPSVAATLGFGAYMSKAHGIEPNIRTGVRTITTGGPITYST
YCKFLADGGCSGGAYDIIICDECHSTDSTTILGIGTVLDQAETAGARL
VVLATATPPGSITVPHPNIEEVALSNTGEIPFYGKAIPIEAIKGGRHL
IFCHSKKKCDELAAKLTGLGLNAVAYYRGLDVSVIPTSGDVVVVATDA
LMTGFTGDFDSVIDCNTCVTQTVDFSLDPTFTIETTTLPQDAVSRAQR
RGRTGRGRSGIYRFVTPGERPSGMFDSSVLCECYDAGCAWYELTPAET
SVRLRAYLNTPGLPVCQDHLEFWESVFTGLTHIDAHFLSQTKQAGDNL
PYLVAYQATVCARAQAPPPSWDQMWKCLIRLKPTLHGPTPLLYRLGAV
QNEVTLTHPITKYIMACMSADLEVVTSTWVLVGGVLAALAAYCLTTGS
VVIVGRIILSGRPAVIPDREVLYQEFDEMEECASHLPYIEQGMQLAEQ
FKQKALGLLQTATKQAEAAAPVVESKWRALEVFWAKHMWNFISGIQYL
AGLSTLPGNPAIASLMAFTASITSPLTTQNTLLFNILGGWVAAQLAPP
SAASAFVGAGIAGAAVGSIGLGKVLVDILAGYGAGVAGALVAFKVMSG
EMPSTEDLVNLLPAILSPGALVVGVVCAAILRRHVGPGEGAVQWMNRL
IAFASRGNHVSPTHYVPESDAAARVTQILSSLTITQLLKRLHQWINED
CSTPCSGSWLKDVWDWICTVLSDFKTWLQSKLLPRLPGLPFLSCQRGY
KGVWRGDGIMQTTCPCGAQITGHVKNGSMRIVGPKTCSNTWHGTFPIN
AYTTGPCTPSPAPNYSRALWRVAAEEYVEVTRVGDFHYVTGMTTDNVK
CPCQVPAPEFFTEVDGVRLHRYAPVCKPLLREEVVFQVGLNQYLVGSQ
LPCEPEPDVAVLTSMLTDPSHITAETAKRRLARGSPPSLASSSASQLS
APSLKATCTTHHDSPDADLIEANLLWRQEMGGNITRVESENKVVILDS
FDPIRAVEDEREISVPAEILRKPRKFPPALPIWARPDYNPPLLESWKD
PDYVPPVVHGCPLPSTKAPPIPPPRRKRTVVLTESTVSSALAELATKT
FGSSGSSAVDSGTATGPPDQASDDGDKGSDVESYSSMPPLEGEPGDPD
LSDGSWSTVSGEAGEDVVCCSMSYTWTGALITPCAAEESKLPINPLSN
SLLRHHSMVYSTTSRSASLRQKKVTFDRLQVLDDHYRDVLKEMKAKAS
TVKARLLSIEEACKLTPPHSAKSKFGYGAKDVRSLSSRAVNHIRSVWE
DLLEDTETPIDTTIMAKNEVFCVQPEKGGRKPARLIVFPDLGVRVCEK
MALYDVVSTLPQAVMGPSYGFQYSPGQRVEFLVNTWKSKKCPMGFSYD
TRCFDSTVTENDIRTEESIYQCCDLAPEARQAIRSLTERLYVGGPLTN
SKGQNCGYRRCRASGVLTTSCGNTLTCYLKATAACRAAKLQDCTMLVN
GDDLVVICESAGTQEDAAALRAFTEAMTRYSAPPGDPPQPEYDLELIT
SCSSNVSVAHDASGKRVYYLTRDPTTPLARAAWETVRHTPVNSWLGNI
IMYAPTLWARMILMTHFFSILLAQEQLEKALDCQIYGACYSIEPLDLP
QIIERLHGLSAFSLHSYSPGEINRVASCLRKLGVPPLRVWRHRARSVR
AKLLSQGGRAATCGKYLFNWAVKTKLKLTPIPAASQLDLSGWFVAGYN
GGDIYHSLSRARPRWFMLCLLLLSVGVGIYLLPNR).

Furthermore, the full-length amino acid sequence of the certain genome protein of the HCV D89815 strain is shown in SEQ ID NO: 16

(MSTNPKPQRKTKRNTNRRPQDVKFPGGGQIVGGVYLLPRRGPRLGVR
ATRKTSERSQPRGRRQPIPKARRPEGRTWAQPGYPWPLYGNEGLGWAG
WLLSPRGSRPSWGPNDPRRRSRNLGKVIDTLTCGFADLMGYIPLVGAP
LGGAARALAHGVRVLEDGVNYATGNLPGCSFSIFLLALLSCLTIPASA
YEVRNVSGIYHVTNDCSNSSIVYEAADVIMHAPGCVPCVRENNSSRCW
VALTPTLAARNASVPTTTLRRHVDLLVGTAAFCSAMYVGDLCGSVFLI
SQLFTFSPRRHETVQDCNCSIYPGHVSGHRMAWDMMMNWSPTAALVVS
QLLRIPQAVMDMVAGAHWGVLAGLAYYSMVGNWAKVLIVMLLFAGVDG
HTRVTGGVQGHVTSTLTSLFRPGASQKIQLVNTNGSWHINRTALNCND
SLKTGFLAALFYTHKFNASGCPERMASCRSIDKFDQGWGPITYAQPDN
SDQRPYCWHYAPRQCGIVPASQVCGPVYCFTPSPVVVGTTDRFGAPTY
NWGDNETDVLLLNNTRPPHGNWFGCTWMNSTGFTKTCGGPPCNIRGVG
NNTLTCPTDCFRKHPDATYTKCGSGPWLTPRCLVDYPYRLWHYPCTVN
FTIFKVRMYVGGVEHRLDAACNWTRGERCDLEDRDRAELSPLLLSTTE
WQILPCSYTTLPALSTGLIHLHQNIVDIQYLYGIGSAVVSIAIKWEYV
VLLFLLLADARVCACLWMMLLIAQAEAALENLVVLNAASVVGAHGMLP
FFMFFCAAWYMKGRLVPGAAYAFYGVWPLLLLLLALPPRAYAMDREMV
ASCGGGVFVGLALLTLSPYCKVFLARLIWWLQYFITKAEAHLQVSLPP
LNVRGGRDAIILLMCAVHPELIFDITKLLLSILGPLMVLQASLIRVPY
FVRAQGLIRACMLVRKAAGGHYVQMAFVKLAALTGTYVYDHLTPLQDW
AHVGLRDLAVAVEPVVFSAMETKVITWGADTAACGDIISGLPVSARRG
KEILLGPADSFEGQGWRLLAPITAYSQQTRGLLGCIITSLTGRDKNQV
EGEVQVVSTAKQSFLATCVNGACWTVEHGAGSKTLAAAKGPITQMYTN
VDQDLVGWPAPPGARSLTPCTCGSSDLYLVTRHADVIPVRRRGDSRGS
LLSPRPISYLKGSSGGPLLCPSGHVVGIFRAAVCTRGVAKAVDFIPVE
SMETTMRSPVFTDNSTPPAVPQTFQVAHLHAPTGSGKSTKVPAAYAAQ
GYMVLVLNPSVAATLGFGAYMSKAHGIDPNIRTGVRTITTGAPITYST
YGKFLADGGCSGGAYDIIICDECHSTDSTSILGIGTVLDQAETVGARF
VVLATATPPGSITFPHPNIEEVPLANTGEIPFYAKTIPTEVIRGGRHL
IFCHSKKKCDELPAKLSALGLNAVAYYRGLDVSVIPASGDVVVVATDA
LMTGFTGDFDSVIDCNTCVTQTVDFSLDPTFTIETTTVPQDAVSRTQR
RGRTGRGRRGIYRFVTPGERPSAMFDSSVLCECYDAGCAWYELTPAET
SVRLRAYLNTPGLPVCQDHLEFWESVFTGLTHIDAHFLSQTKQAGDNF
PYLVAYQATVCARAKAPPPSWDQMWKCLIRLKPTLHGPTPLLYRLGAV
QNEVTLTHPITKYIMACMSADLEVVTSTWVLVGGVLAALAAYCLTTGS
VVIVGRIILSGRPAVIPDREVLYQEFDEMEECASHLPYIEQGMQLAEQ
FKQKALGLLQTATKQAEAAAPVVESKWRALETFWAKHMWNFISGIQYL
AGLSTLPGNPAIASLMAFTASITSPLATQYTLLFNILGGWVAAQLAPP
SAASAFVGAGIAGAAVGSIGLGKVLVDILACYGAGVAGALVAFKVMSG
DMPSTEDLVNLLPAILSPGALVVGVVCAAILRRHVGPGEGAVQWMNRL
IAFASRGNHVSPTHYVPESDAAARVTQILSNLTITQLLKRLHQWINED
CSTPCSGSWLRDVWDWICTVLADFKTWLQSKLLPRLPGVPFFSCQRGY
KGVWRGDGIMYTTCPCGAQITGHVKNGSMRIVGPRTCSNTWHGTFPIN
AYTTGPCTPSPAPNYSRALWRVAAEEYVEVTRVGDFHYVTGMTTDNVK
CPCQVPAPEFFTELDGVRLHRYAPACKPLLRDEVTFQVGLNQYTVGSQ
LPCEPEPDVTVVTSMLTDPSHITAEAARRRLARGSPPSLAGSSASQLS
ALSLKATCTTHHGAPDTDLIEANLLWRQEMGGNITRVESENKIVILDS
FEPLRAEEDEREVSAAAEILRKTRKFPAAMPVWARPDYNPPLLESWKN
PDYVPPVVHGCPLPPTKAPPIPPPRRKRTVVLTESTVSSALAELATKT
FGGSGSSAVDSGTATGPPDQASAEGDAGSDAESYSSMPPLEGEPGDPD
LSDGSWSTVSEEASEDVVCCSMSYTWTGALITPCAAEESKLPINALSN
PLLRHHNMVYSTTSRSASLRQKKVTFDRMQVLDDHYRDVLKEMKAKAS
TVKAKLLSVEEACKLTPPHSAKSKFGYGAKDVRSLSSRAVNHIRSVWK
DLLEDTDTPIQTTIMAKNEVFCVQPEKGGRKPARLIVFPDLGVRVCEK
MALYDVVSTLPQAVMGSSYGFQYSPKQRVEFLVNTWKAKKCPMGFSYD
TRCFDSTVTENDIRVEESIYQCCDLAPEARQAIRSLTERLYIGGPMTN
SKGQNCGYRRCRASGVLTTSCGNTLTCYLKAAAACRAAKLQDCTMLVC
GDDLVVICDSAGTQEDAASLRVFTEAMTRYSAPPGDPPQPEYDLELIT
SCSSNVSVAHDASGKRVYYLTRDPTTPLARAAWETARHTPVNSWLGNI
IMYAPTLWARMILMTHFFSILLAQEQLEKALDCQIYGATYSIEPLDLP
QIIQRLHGLSAFSLHSYSPGEINRVASCLRKLGVPPLRVWRHRARSVR
AKLLSQGGRAATCGKYLFNWAVKTKLKLTPIPEASQLDLSGWFVAGYS
GGDIYHSLSRARPRWFMWCLLLLSVGVGIYLLPNR).

### <Cytotoxic T-Cell Induction Experiment>

The following experiment was carried out using a human blood sample.

### (1) Activation of Cytotoxic T-Cells

Blood was sampled from a hepatitis C patient or a healthy individual from whom informed consent had been obtained in advance.

To blood cells (Peripheral blood mononuclear cells: PBMCs) sown on 96 well plates peptides of SEQ ID NOS: 1 to 14 were added weekly for 5 weeks so as to give final concentrations of 1 μM, and cytotoxic T-cells were activated in media to which was added 10% FCS and 5 ng/mL of IL-2, thus giving T-cell samples.

### (2) Treatment of ⁵¹Cr-Labeled Cells

Cells were labeled with ⁵¹Cr by adding the sodium salt of ⁵¹Cr (sodium chromate) to C1R-A24 cells and T2-A24 cells (human B/T cultured cell strain having no TAP peptide transporter and expressing the HLA-A*2402 gene: supplied by Dr. Tsuboi, Faculty of Medicine, Osaka University) at 37° C. or 26° C. for 1 hour. Peptides of SEQ ID NOS: 1 to 14 were added thereto so as to make final concentrations of 1 μM, thus forming target cells having each peptide binding to the surface of the cells.

### (3) Damaging of Labeled Cells

After the cytotoxic T-cells and the target cells prepared above were mixed at an E/T ratio (effector to target) of 10 to 20 and allowed to stand at 37° C. for 3.5 hours, the radioactivity of ⁵¹Cr released in the culture supernatant was measured. The influence of nonspecific activity that does not depend on the action of cytotoxic T-cells was subtracted, and the cytolytic activity against the labeled cells was calculated.

### <Results of Induction Experiment>

FIG. 2 and FIG. 3 show the results of cytotoxic T-cell induction experiments of peptides with each amino acid sequence in T-cell samples cultured in separate media. In the figures, data of blood samples derived from 8 people (or 7 people) are shown in ‘series 1’ to ‘series 8 (or series 7)’.

With regard to the sequences of SEQ ID NOS: 1 to 13, blood from any one of the 8 people showed a capability of inducing cytotoxic T-cells. In contrast, with regard to the sequence of SEQ ID NO: 14, none of the blood samples showed a capability of inducing cytotoxic T-cells. This trend was seen in both FIG. 2 and FIG. 3 in common.

It is surmised that the reason why the same capability for inducing cytotoxic T-cells was not shown for blood from all of the 8 people by the amino acid sequences of SEQ ID NOS: 1 to 13 in both FIG. 2 and FIG. 3 is that, although there is no difference in binding to an HLA molecule between individuals, since the T-cell repertoire of an individual differs depending on individual genetic background or past infection history, there is a possibility of individual differences in peptide reactivity appearing.

Therefore, FIG. 2 and FIG. 3 suggest that, from the viewpoint of responding to a large number of patients, it is preferable to use a plurality of types of amino acid at the same time.

### EXAMPLE 2

### <Damaging Activity Targeted at HCV-Expressing Cells>

### (1) Preparation of HCV Genome-Expressing Cells

RzM6 cells were formed by transfecting HepG2, which is a human liver cell strain, with an HCV gene that had been modified so as to be able to conditionally induce expression of an HCV1b full genome (AY045702) in the presence of Cre (Activation of the CK1-CDK-Rb-E2F pathway in full genome hepatitis C virus-expressing cells. Tsukiyama-Kohara et al., J. Biol. Chem. 279, 14531-14541, 2004, RzM6 being supplied by Dr. Michinori Obara, Tokyo Metropolitan Institute of Medical Science).

In the HCV expression method, since the same RzM6 cells are doubly transfected with the Cre gene inserted in a conditional expression cassette arranged downstream of a Tamoxifen-inducible promoter, by adding tamoxifen, expression of Cre is induced, and as a result expression of the HCV gene can be induced. The RzM6 cells for which expression of the HCV gene was induced were labeled with ⁵¹Cr by the same method as for the C1R-A24 cells, thus serving as target cells.

### (2) Damage to HCV Genome-Expressing Cells

The cytotoxic T-cells prepared above and RzM6 target cells or HepG2 cells with no HCV introduced as a control were mixed at an E/T ratio of 10 and allowed to stand at 37° C. for 3 hours, and the radioactivity of ⁵¹Cr released into the culture supernatant was measured. The influence of nonspecific activity that does not depend on the action of cytotoxic T-cells was subtracted, and the cytolytic activity against target cells was calculated.

### <Results of Cytolysis Assay Against HCV Genome-Expressing Cells>

FIG. 4 shows the results of a cytolysis assay against HCV genome-expressing RzM6 cells with a cell line which was grown from peripheral blood T-cells were taken from different people and stimulated by the peptides of each of the amino acid sequences so as to increase reactive T-cells. Each bar of the graph shows the cytolytic activity of individual cell line grown as above against RzM6 cells.

With regard to the sequences of SEQ ID NOS: 1, 2, 4, 5, 7, 8, 9, 10, 12, and 13, a cytolytic activity specific to HCV-expressing cells by the cytotoxic T-cells was exhibited for the blood from any one of the patients.

### EXAMPLE 3

### <Animal Model Used>

HCV has the property that it does not replicate in anything other than humans or chimpanzees. However, when chimpanzees are used as an animal model, although some develop chronic hepatitis, there is a tendency for it to be cured as acute hepatitis, and the pathophysiology is not the same as for human beings. Moreover, a very large expenditure is required, it takes at least one year for the onset of chronic hepatitis, it is ethically difficult to sacrifice the chimpanzee after use, and it is in practice impossible to carry out an infection experiment in a normal research organization. As a substitute animal model, a system in which a small animal such as a mouse is infected using an expression vector that has been modified so as to express the HCV gene is utilized.

### <Experimental Materials>

### (1) HCV Transgenic Mouse (CN2-29)

A transgenic mouse that conditionally expresses only a portion (nt: 294-3435) corresponding to the front ⅓ of the HCV genome, produced by Dr. Michinori Obara, Tokyo Metropolitan Institute of Medical Science, is used (reference: Efficient conditional transgene expression in Hepatitis C virus cDNA transgenic mice mediated by the Cre/loxP system. Wakita et al., J. Biol. Chem. 273, 9001-9006, 1998.).

(2) Mouse in which Good T-Cell Induction Occurs (CBF1)

The CN2-29 mouse has a BALB/c mouse (H-2^{d}) background, and K^{d}, D^{d}, L^{d}-binding peptides are target epitopes. Mice were immunized using these binding peptides, and induction of T-cells was attempted, but only weak cytotoxicity was induced. Since one with good T-cell induction was found for the D^{b}-binding peptide, if an F1 mouse (CBF1) produced by crossbreeding the CN2-29 mouse with a C57BL/6 (abbreviated to B6, H-2^{b}) mouse is used as a host, in vivo cytotoxicity can be examined.

### (3) EGFP Transgenic Mouse

A transgenic mouse having a B6 mouse genetic background and expressing EGFP (jellyfish-derived fluorescent gene) ubiquitously in the whole body.

### (4) Cre Expression Vector (BCre)

An expression vector formed by incorporating the Cre gene into a bovine papilloma virus-based expression vector called BCMGSneo (reference: Establishment of mouse cell lines which constitutively secrete large quantities of interleukin 2, 3, 4 or 5, using modified cDNA expression vectors. Karasuyama et al. Eur. J. Immunol. 18, 97-104, 1988). Since multiple copies of this vector are present within a cell as episomes, it is possible to increase the number of copies of Cre gene per cell. Furthermore, since it increases as an episome, it expresses without being affected by chromatin structure.

### (5) Mouse HCV-Specific T-Cell Antigen Peptide

When HCV specific D^{b}-binding peptides were searched for using a mouse MHC class I-binding peptide prediction program developed by Udaka et al., Kochi University (reference: An automated prediction of MHC class I-binding peptides based on positional scanning with peptide libraries. Udaka, et al. Immunogenetics. 51, 816-828, 2000.), CD649 (log Kd=−7.58) induced cytotoxic T-cells (CTL) well in the CBF1 mouse.

(6) Whole-Cell Bordetella pertussis Vaccine

With regard to the Bordetella pertussis vaccine used for human beings, there are purified cell vaccine and whole-cell vaccine; among them the whole-cell vaccine is available from Bio Farma of Indonesia, and it is confirmed that if the whole-cell vaccine is administered together with a peptide, the induction of cytotoxic T-cells were augmented remarkably in mice.

### (7) Hydrodynamic Injection (HDI) Method

A method in which an anesthetized mouse is intravenously injected in about 5 sec with a large volume of physiological solution (about 1.5 mL) in which plasmid DNA is dissolved and the venous pressure is temporarily raised, thus increasing the efficiency of plasmid uptake. The venous pressure becomes particularly high in tissues such as the liver and spleen where venous blood is easily retained, and since part of the cell membrane is physically torn or intake an extracellular solution is accelerated due to pinocytosis, the plasmid is efficiently taken up into the cytoplasm of liver cells.

### <Hepatitis Onset Experiment>

A CBF1 mouse is intradermally injected with CD649 and whole-cell Bordetella pertussis vaccine once a week a total of 4 times, thus inducing cytotoxic T-cells (CTL), Cre gene is then expressed using the HDI method, and expression of the HCV gene possessed by the CBF1 mouse is induced. In this mouse, whether or not hepatitis has occurred naturally is checked by examining liver tissue and measuring serum AST and ALT enzyme (group of enzymes released from damaged liver cells) values. Furthermore, the degrees of lymphocyte infiltration and tissue damage are investigated by histological tissue examination.

### <In Vivo CTL Infiltration Experiment>

An EGFP transgenic mouse is intradermally injected with CD649 and whole-cell Bordetella pertussis vaccine once a week a total of 4 times, thus inducing cytotoxic T-cells (CTL), and immunocytes are then taken out from the spleen and subjected to peptide stimulation twice in vitro, thus increasing the proportion of CD649-specific CTL. A CBF1 mouse in which expression of the HCV gene has been induced by the above-mentioned hepatitis onset experiment is intravenously injected with the above CTL, a few days later the liver is examined using a fluorescent microscope, and the number of EGFP positive cells that have infiltrated the liver is compared with the case of a mouse intravenously injected with an empty vector.

### <Experiment to Examine In Vivo Tissue Damage>

A vector that simultaneously expresses the Cre gene and EGFP is created and prepared so that only liver cells that induce expression of the HCV gene emit EGFP fluorescence. In the above-mentioned hepatitis onset experiment, by using this Cre-EGFP expression vector, a histology in which the liver is infiltrated by cytotoxic T-cells (CTL) and EGFP expressing cells (i.e. HCV gene expressing cells) are preferentially damaged, and the like, is observed.

### <Confirmation of Safety of Peptide Immunotherapy>

Confirm that even when a mouse is hyperimmunized with CD649 peptide and whole-cell Bordetella pertussis vaccine, normal tissue is not attacked. Subsequently, in the CN2-29 mouse in which expression of HCV has been induced, examine whether or not attack by CTL causes hepatic failure.

The present invention is explained above by reference to Examples. These Examples are only illustrated as examples, and a person skilled in the art will understand that various modification examples are possible, and such modification examples are included in the scope of the present invention.

1. A method for inducing cytotoxic T-cells, the method comprising:
generating a target cell for cytotoxic T-cells on which surface HLA molecules are bound by one or more types of amino acid sequence selected from the group consisting of SEQ ID NOS: 1 to 13 and consisting of not less than 8 and not more than 11 amino acid residues, or a peptide derived from a precursor thereof.

2. The method as set forth in claim 1,
wherein the peptide or the peptide derived from a precursor thereof binds to a human HLA-A24 molecule on the surface of the target cell.

3. A cytotoxic T-cell inducer comprising at least one of a peptide and a precursor thereof, the peptide comprising one or more types of amino acid sequence selected from the group consisting of SEQ ID NOS: 1 to 13, consisting of not less than 8 and not more than 11 amino acid residues, and binding to an HLA molecule on the surface of a cell that is a target of a cytotoxic T-cell.

4. A pharmaceutical composition for the treatment of a disease that is caused by hepatitis C virus, the composition comprising at least one of a peptide and a precursor thereof, the peptide comprising one or more types of amino acid sequence selected from the group consisting of SEQ ID NOS: 1 to 13, consisting of not less than 8 and not more than 11 amino acid residues, and binding to an HLA molecule on the surface of a cell infected with hepatitis C virus.

5. A vaccine used for the prevention or treatment of a disease that is caused by hepatitis C virus, the vaccine comprising at least one of a peptide and a precursor thereof, the peptide comprising one or more types of amino acid sequence selected from the group consisting of SEQ ID NOS: 1 to 13, consisting of not less than 8 and not more than 11 amino acid residues, and binding to an HLA molecule on the surface of a cell infected with hepatitis C virus.
